(19) United States
(12) Patent Application Publication
(10) Pub. No.: US 2005/0229431 A1
(43) Pub. Date: Oct. 20, 2005

(54) Sole for shoes

(75) Inventor: Stefano Gerlin, Pieve Di Soligo (IT)

Correspondence Address: MODIANO & ASSOCIATI, Via Meravigli, 16, Mikan, 20123, IT

(73) Assignee: Maurizio CONTOLINI

(21) Appl. No.: 11/139,485

(22) Filed: May 31, 2005

(30) Foreign Application Priority Data
Apr. 16, 2004 (IT) VR2004A000062

(51) Int. Cl. A43B 13/00; A43B 13/12; A43B 13/14; A61F 13/00; A43B 13/38; A61F 15/00; A43B 23/00

(52) U.S. Cl. 36/25 R; 36/30 R

(57) ABSTRACT

A sole for shoes comprising a sole base body, the sole base body having at least one cutout that is closed at least partially by at least one multilayer sheet insert; the insert has, in its lower part intended to be directed toward the ground, a layer made of rubber material, which is molded directly by direct microinjection onto the remaining part of the multilayer sheet insert.

### BACKGROUND OF THE INVENTION

As is known, soles adapted to allow better comfort and relief in walking are manufactured in the field of shoes. Among some of the known solutions, mention can be made for example of the manufacture of soles that have microperforations that are a direct component of the structure that constitutes the sole and on which breathable insoles rest. These solutions accordingly suffer particular manufacturing difficulties, with high costs and in many cases with a reduced flexibility of the sole.

### SUMMARY OF THE INVENTION

The aim of the present invention is to eliminate the drawbacks noted above by providing a sole for shoes that thanks to its particular constructive characteristics is more elastic and flexible, so as to allow easy and comfortable walking.

Within this aim, an important object of the present invention is to provide a sole for shoes that can ensure excellent waterproofing.

Another object of the present invention is to provide a sole for shoes that can be manufactured with reduced manufacturing costs, so as to be competitive also from a merely economical standpoint.

This aim and these and other objects that will become better apparent hereinafter are achieved by the sole for shoes according to the invention, which comprises a sole base body and is characterized in that said sole base body has at least one cutout that is closed at least partially by at least one multilayer sheet-like insert, which has, in its lower part intended to be directed toward the ground, a layer made of rubber-like material or the like, which has at least one through opening and is molded directly by direct microinjection onto the remaining part of said multilayer sheet-like insert.

### BRIEF DESCRIPTION OF THE DRAWINGS

Further characteristics and advantages of the invention will become better apparent from the description of some preferred but not exclusive embodiments of the sole for shoes according to the invention, illustrated by way of non-limiting example in the accompanying drawings, wherein:

FIG. 1 is a schematic perspective top view of the sole for shoes according to the invention;

FIG. 2 is an exploded perspective view of the two parts that constitute the sole;

FIG. 3 is a bottom perspective view of the sole according to the invention;

FIG. 4 is a bottom perspective view of the multilayer sheet-like insert;

FIG. 5 is an exploded bottom perspective view of the multilayer sheet-like insert;

FIG. 6 is a sectional view, taken along the line VI-VI of FIG. 4;

FIG. 7 is an enlarged-scale sectional view, taken along the line VII-VII of FIG. 4;

FIG. 8 is a partially cutout view of the multilayer sheet-like insert;

FIG. 9 is an exploded perspective view of a second embodiment of the sole according to the invention;

FIG. 10 is a top perspective view of the sole base body according to the second embodiment of FIG. 9;

FIG. 11 is an exploded bottom perspective view of the multilayer sheet-like insert again according to the second embodiment of FIG. 9;

FIG. 12 is a bottom perspective view of the multilayer sheet-like insert of FIG. 11 according to the second embodiment of FIG. 9;

FIG. 13 is a bottom perspective view of the second embodiment of FIG. 9;

FIG. 14 is an exploded bottom perspective view of a third embodiment of the invention;

FIG. 15 is a bottom view of the third embodiment of FIG. 14;

FIG. 16 is an exploded perspective view of a fourth embodiment of the invention;

FIG. 17 is a bottom view of the embodiment of FIG. 16.

### DESCRIPTION OF THE PREFERRED EMBODIMENTS

In the examples of embodiments that follow, individual characteristics, given in relation to specific examples, may actually be interchanged with other different characteristics that exist in other examples of embodiments.

Moreover, it is noted that anything found to be already known during the patenting process is understood not to be claimed and to be the subject of a disclaimer.

With reference to the figures, the sole for shoes according to the invention, generally designated by the reference numeral 1, generally comprises a base body 2 of the sole and at least one multilayer insert 3.

It should be noted that the base body 2 of the sole can be conveniently made of die-cast material or of any other suitable material, both natural, such as for example leather (as in the embodiment illustrated merely by way of example in FIGS. 14 and 15), and synthetic.

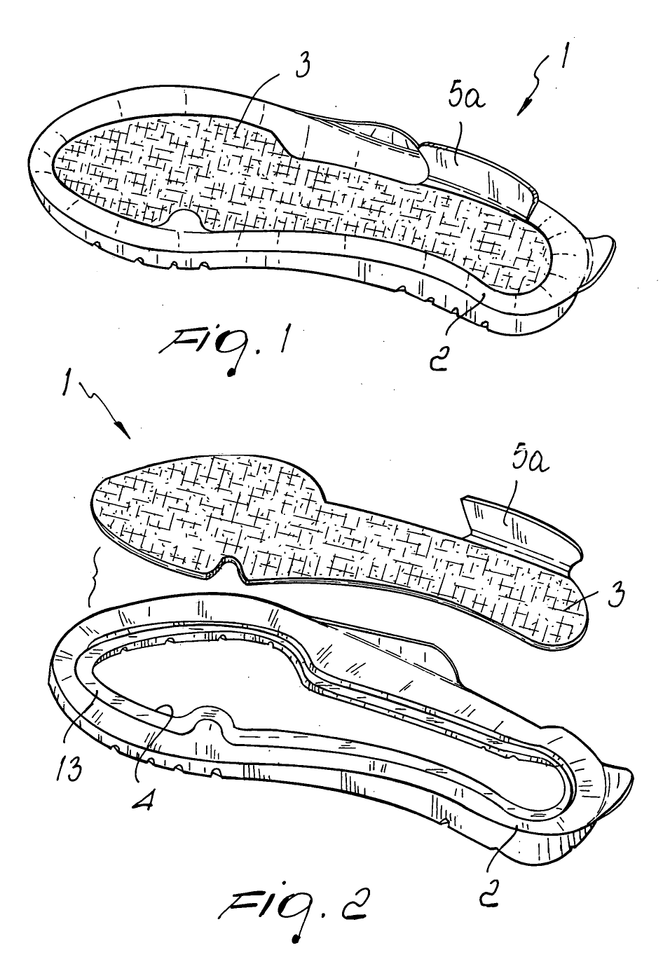

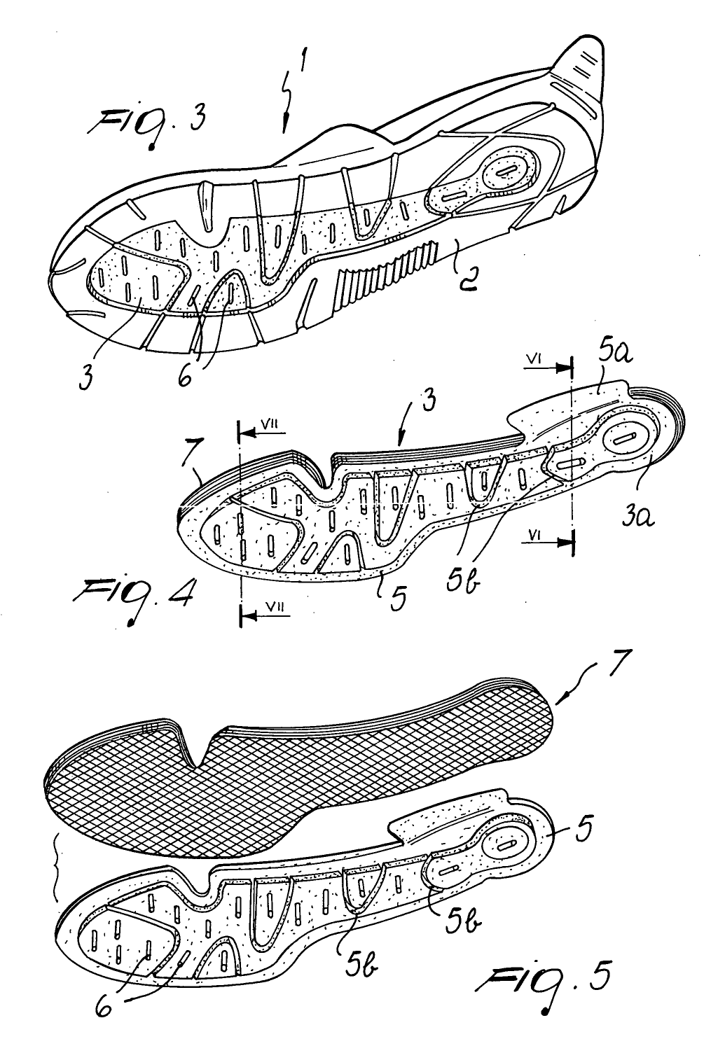

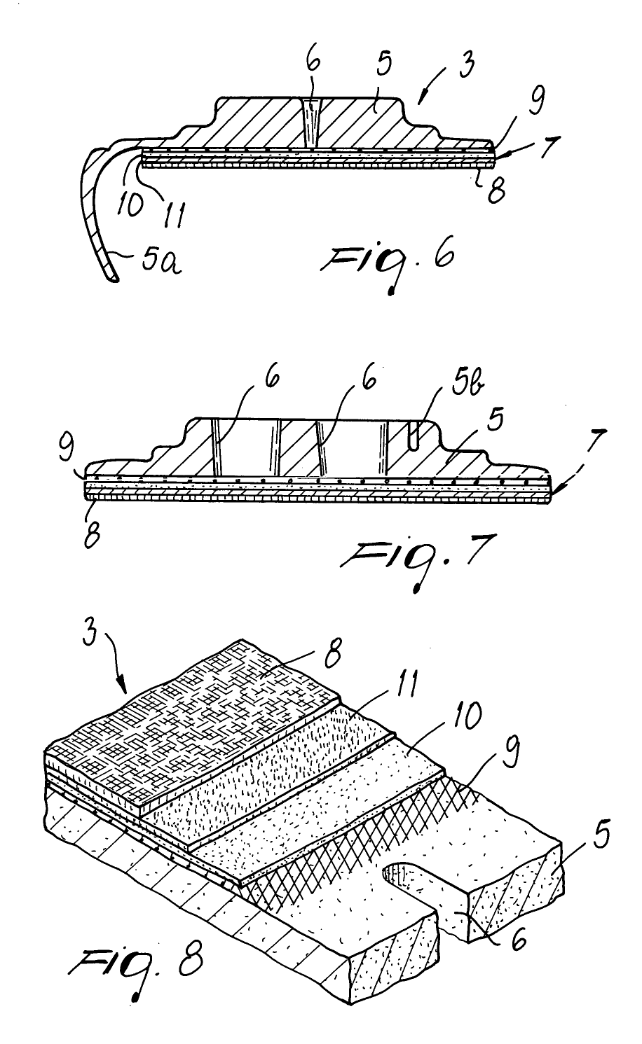

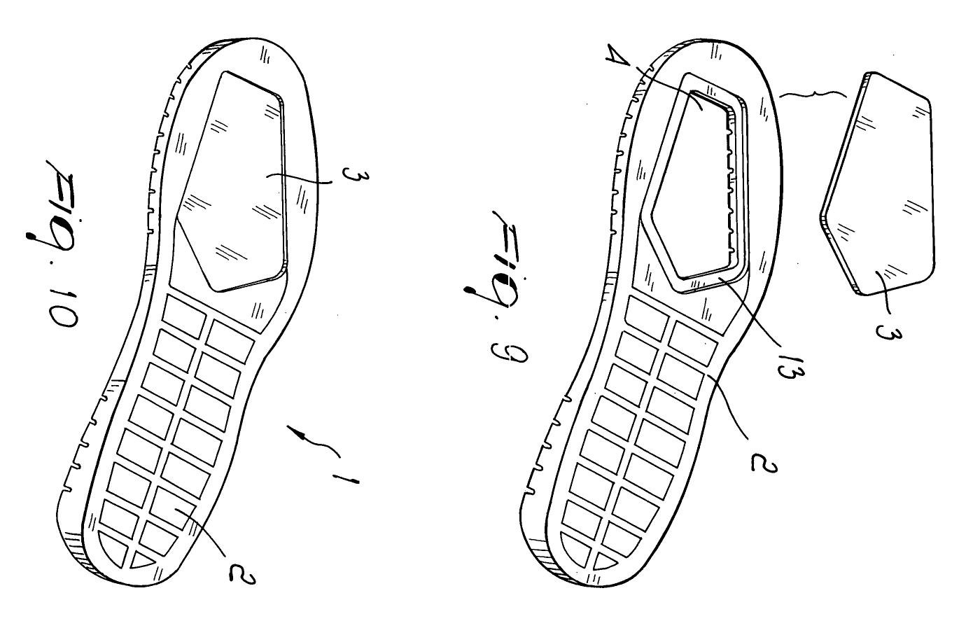

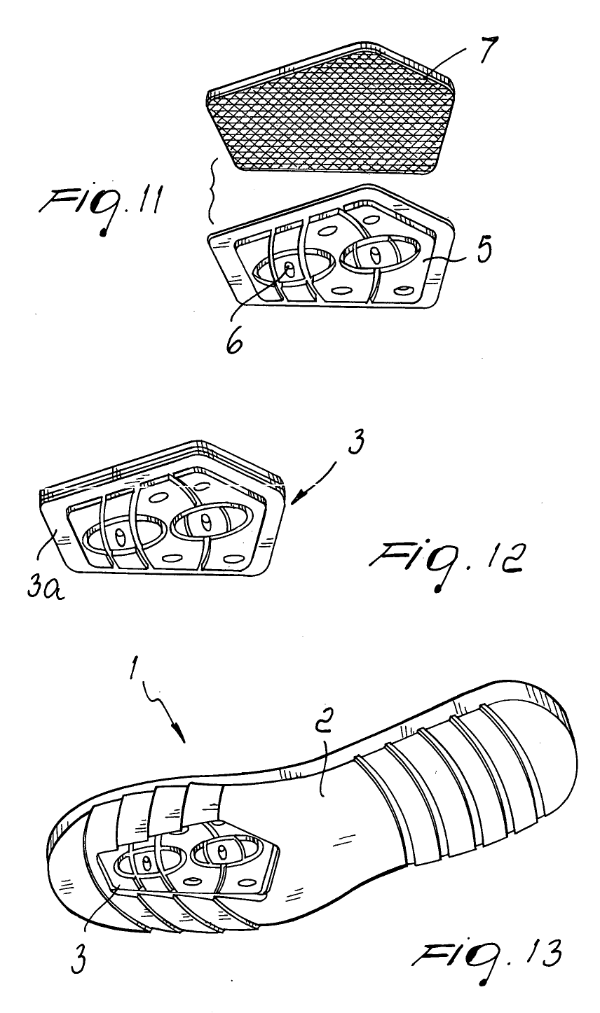

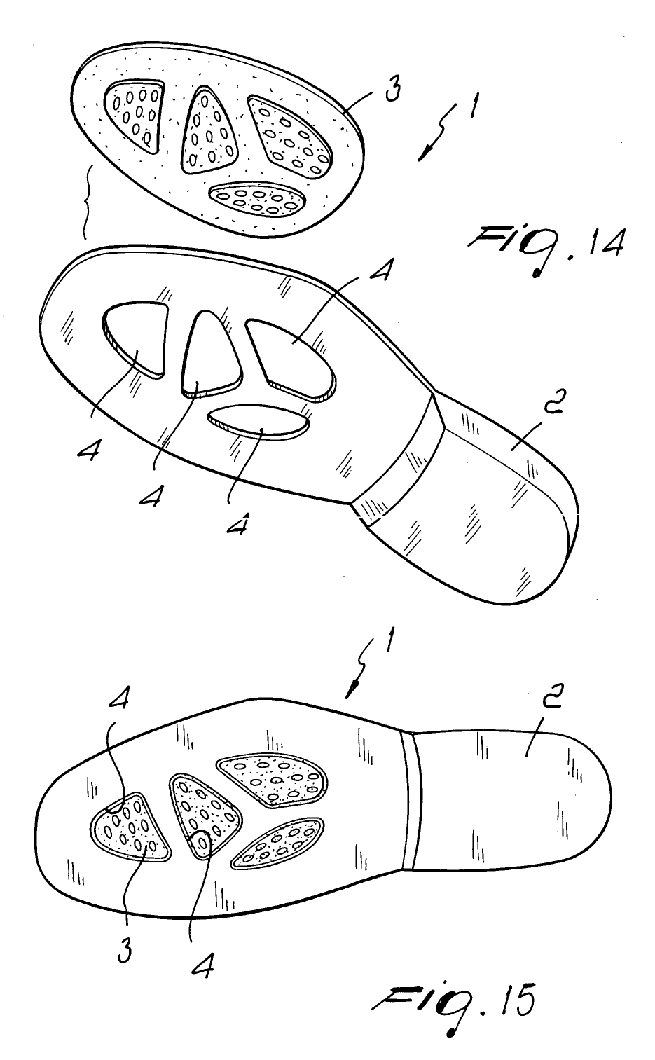

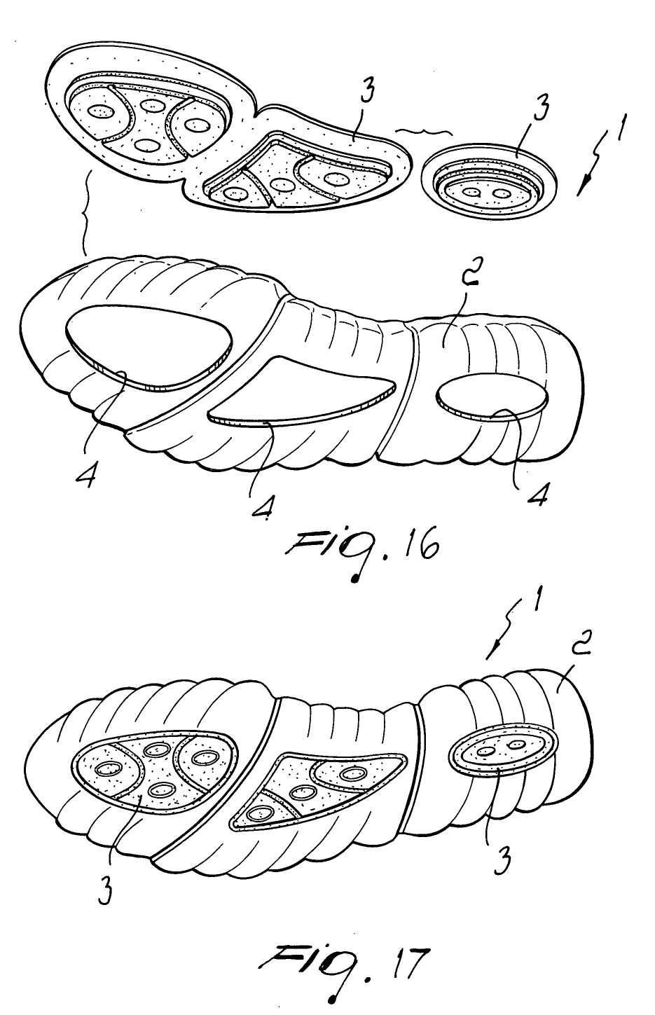

According to the invention, the base body 2 of the sole has at least one cutout 4, which can be arranged advantageously in any part of the base body 2, i.e., in the front and/or central and/or rear part of the sole base body 2 (as shown in FIG. 10 or FIG. 16), or can run substantially along the entire extension of the sole base body 2 (as shown in FIG. 2). The cutout 4 can be provided for example directly during the molding of the sole base body 2 or can be provided by cutting or by means of any other system that is deemed more appropriate. Moreover, the cutout 4 can have a polygonal, oval, elliptical, circular shape or any other more appropriate shape.

Also according to the invention, the cutout 4 is closed at least partially by at least one multilayer sheet-like insert 3, which has, in its lower part intended to be directed toward the ground, a layer 5, which is made of rubber-like or similar material (for example an elastomer) and is provided with at least one through cutout 6 that passes through its thickness. In particular, such layer 5 is molded directly by direct microinjection onto the remaining part of the multilayer sheet-like insert 3.

In this manner, the layer 5 effectively grips the remaining part of the multilayer sheet-like insert 3.

Merely by way of example, the layer 5 can be made of an elastomer, such as for example the material known by the trade-name APILON 3®, manufactured by the Italian company API S.p.A. and substantially constituted by a thermoplastic polyurethane (TPU).

Advantageously, the layer 5 is provided with a plurality of through openings 6, which are distributed substantially along its entire extension and conveniently have an elongated transverse cross-section with walls that diverge toward the ground. In a possible embodiment, the layer 5 is affected by one or more surface grooves 5b.

It should be noted that if there are multiple cutouts 4 in the sole base body 2, each cutout is advantageously at least partially closed by at least one respective multilayer sheet-like insert 3.

In greater detail, in order to obtain the multilayer sheet-like insert 3, the layer 5, which in practice acts as an insole, is assembled by direct microinjection on a sandwich-like multilayer element 7.

Advantageously, the sandwich-like multilayer element 7 in turn comprises at least one temperature control layer 8, which is designed to stabilize the temperature of the foot inside the shoe by providing adequate thermal insulation.

Conveniently, the temperature control layer 8 is constituted by a fabric made of synthetic or natural hollow fibers. Merely by way of example, the hollow-fiber fabric marketed by the Italian company Tintotex and in particular the fabric known by the trade name Meryl Nexten®, of the Nylstar company, has been found to be suitable for the purpose.

Advantageously, the sandwich-like multilayer element 7 further has at least one mechanical protection layer 9, which is designed to provide the multilayer sheet-like insert 3 with a valid mechanical strength, particularly resistance to abrasion, cutting, piercing and/or tearing. In particular, it is preferable to provide the mechanical protection layer 9 by means of a very fine mesh, which can be made of metal, carbon fiber or polyamide, such as for example the one manufactured by the Italian company Mosocca, or by means of a fabric made of aramid fiber or of any other suitable type.

In order to allow vapor permeation of the foot toward the outside of the shoe without compromising waterproofing of the sole according to the invention, the sandwich-like multilayer element 7 conveniently comprises at least one breathable and water-repellent layer 10, which is advantageously constituted by a microporous membrane, such as for example the membrane known commercially as Fit-Top®, of the PDG company.

In order to ensure satisfactory comfort for the user's foot, the sandwich-like multilayer element 7 can advantageously also have one or more layers of padding 11, preferably constituted by a felt made of non-woven fabric.

It should also be added that the multilayer sheet-like insert 3 can be conveniently completed by a moisture absorption layer (not shown), which is located in its upper part, which is intended to be directed toward the foot and make contact therewith, and is constituted for example by a mesh made of vegetable fiber, such as cotton or the like, or synthetic fiber, such as polyester or other equivalent material.

The layers 8, 9, 10, 11 of the sandwich-like sheet element 7 are assembled to each other for example by mutual adhesive bonding on their peripheral portion or internally by spot gluing.

The arrangement of the layers 8, 9, 10, 11 within the thickness of the multilayer sheet-like insert may be any.

It is in any case preferable that the mechanical protection layer 9 be arranged adjacent to, or in contact with, the layer 5.

One possible arrangement of the layers 8, 9, 10, 11 is shown in particular, merely by way of non-limiting example, in FIG. 8.

Preferably, the multilayer sheet-like insert 3 is thinner than the sole base body 2 and conveniently has a shape that matches the shape of the corresponding cutout 4, so as to close it completely.

Advantageously, the sole according to the invention is obtained by separately providing the multilayer sheet-like insert 3 and the sole base body 2 and by then inserting the multilayer sheet-like insert 3 in a seat formed by a lip 13 arranged laterally with respect to the cutout 4, so that for example, the multilayer sheet-like insert 3 can close completely the free part thereof.

Conveniently, the peripheral portion 3a of the multilayer sheet-like insert 3 is joined intimately to the lip 13 formed on the sole base body 2 by adhesive bonding, vulcanization, electric welding of any type or another system deemed appropriate, so as to prevent water from infiltrating through the joints.

The sole base body 2 can also be provided, after providing the multilayer sheet-like insert 3, around the perimeter of the multilayer sheet-like insert 3 by injection molding.

Advantageously, the multilayer sheet-like insert 3 is arranged so as to remain, with respect to the ground contact part of the sole base body 2, at a higher level so as to not rest directly on the ground.

This allows, during walking, to obtain a cyclic deformation of the multilayer sheet-like insert 3 with a reciprocating motion along a direction that is substantially perpendicular to the plane of arrangement of the sole according to the invention, which generates a pumping effect that allows to draw air from the outside toward the inside of the shoe and to expel outwardly the perspiration of the foot from the inside of the shoe, thus facilitating foot breathing through the multilayer sheet-like insert 3 and particularly through the through openings 6 formed in the layer 5.

As can be seen in the figures, the layer 5 can have, for merely aesthetic reasons, a tab 5a that protrudes outwardly from the perimetric rim of the multilayer sheet-like insert 3 and is designed to be arranged on the external surface of a side of the shoe.

All the characteristics of the invention described above as advantageous, convenient or the like may also be omitted or be replaced with equivalents.

The individual characteristics described with reference to general teachings or to particular embodiments may all be present in other embodiments or may replace characteristics in those other embodiments.

The invention thus conceived is susceptible of numerous modifications and variations, all of which are within the scope of the appended claims.

In practice it has been observed that in all its embodiments, the invention has achieved its intended aim and objects, and in particular the fact is stressed that the invention provides a sole with lightweight and reduced rigidity characteristics that is at the same time very strong, ensuring better comfort and relief in walking for the foot.

Another important advantage of the invention occurs if the lower layer, intended to be directed toward the ground, of the or each multilayer sheet-like insert is made of an elastomer. This material in fact has the well-known property of returning to its original state (so-called “memory”) after deformation. Therefore, during walking the lower layer of the multilayer sheet-like insert will tend to assume the shape of the foot, adapting to it, with an obvious improvement in shoe comfort, and then returns to its original shape once the shoe has been removed. This gives the sole according to the invention a considerable degree of nondeformability, regardless of the material of which its base body is made (rubber, leather, thermoresin or other), which after using the shoe resumes its original shape indeed due to the “return” of the elastomer that provides the lower layer directed toward the ground of the or each multilayer sheet-like insert.

In practice, the materials used, so long as they are compatible with the contingent use, as well as the shapes and dimensions, may be any according to requirements.

All the details may further be replaced with other technically equivalent elements.

1. A sole for shoes, comprising a sole base body, wherein said sole base body has at least one cutout that is closed at least partially by at least one multilayer sheet insert, which has, in its lower part intended to be directed toward the ground, a layer made of rubber material, which is molded directly by direct microinjection onto the remaining part of said multilayer sheet insert.

2. The sole for shoes according to claim 1, wherein said layer of rubber or similar material comprises at least one through opening.

3. The sole for shoes according to claim 1, wherein said at least one multilayer sheet insert can be inserted in a seat that delimits said cutout so as to comprise all the free part of said cutout.

4. The sole for shoes according to claim 1, wherein said at least one multilayer sheet insert comprises at least one layer for foot temperature control.

5. The sole for shoes according to claim 4, wherein said at least one temperature control layer comprises a fabric made of hollow fiber.

6. The sole for shoes according to claim 1, wherein said at least one multilayer sheet insert comprises at least one mechanical protection layer.

7. The sole for shoes according to claim 6, wherein said at least one mechanical protection layer comprises a fine mesh.

8. The sole for shoes according to claim 1, wherein said at least one multilayer sheet insert comprises at least one layer made of a water-repellent breathable membrane.

9. The sole for shoes according to claim 1, wherein said at least one multilayer sheet insert comprises at least one layer of padding.

10. The sole for shoes according to claim 9, wherein said at least one padding layer comprises a felt made of non-woven fabric.

11. The sole for shoes according to claim 1, wherein said at least one multilayer sheet insert comprises, in its upper part intended to be directed toward the foot, a moisture absorption layer.

12. The sole for shoes according to claim 11, wherein said moisture absorption layer comprises a mesh made of vegetable or synthetic fiber.

13. The sole for shoes according to claim 1, wherein said sole base body is made of die-cast material or of another suitable natural or synthetic material.

14. The sole for shoes according to claim 1, wherein said at least one cutout lies at a substantially front and/or central and/or rear part of said sole base body or along the extension of said sole base body.

15. The sole for shoes according to claim 1, wherein said at least one cutout is provided directly during the process for molding said sole base body either by cutting or by means of another system.

16. The sole for shoes according to claim 1, wherein said at least one multilayer sheet insert has a shape that corresponds to the shape of the said at least one cutout.

17. The sole for shoes according to claim 1, wherein said at least one multilayer sheet insert is intimately joined to said sole base body, so as to be monolithic therewith, by adhesive bonding, vulcanization, electric welding or other means.

18. The sole for shoes according to claim 1, wherein said at least one multilayer sheet insert is spaced upwardly with respect to the ground contact part of said sole base body, so as to not rest directly on the ground.
